(19) United States
(12) Patent Application Publication
(10) Pub. No.: US 2007/0055096 A1
(43) Pub. Date: Mar. 8, 2007

(54) Sexual stimulation devices and toys with features for playing audio and/or video received from an external source

(76) Inventors: Cheryl J. Berry, St. Louis, MO (US); Jeffrey D. Kleaman, St. Louis, MO (US); Geoffrey Stern, Westport, CT (US)

Correspondence Address: LATHROP & GAGE LC, 2345 GRAND AVENUE, SUITE 2800, KANSAS CITY, MO, 64108, US

(21) Appl. No.: 11/496,537

(22) Filed: Jul. 31, 2006

Related U.S. Application Data
(60) Provisional application No. 60/704,523, filed on Aug. 1, 2005.
(60) Provisional application No. 60/703,983, filed on Jul. 29, 2005.

(51) Int. Cl. A61F 5/00

(52) U.S. Cl. 600/38

(57) ABSTRACT

According to various aspects of the present invention, adult sexual aids, sexual stimulation devices and sex toys, such as vibrators, include means (for example, BLUETOOTH® technology) for communicating with (receiving and/or transmitting) and playing audio and/or video from a remote source, such as a cellular phone, a landline-based phone, the Internet, a personal computer, a cable network, a satellite network, a radio, a television, among other possible remote sources. In one particular aspect, a vibrator includes a BLUETOOTH® module configured to link to a BLUETOOTH® enabled device, such as a cellular phone or a Voice Over IP (VOIP) computer. Aspects of the invention also include methods of using a device of the present invention. In one exemplary implementation, a device of the present invention is used for sexual stimulation while engaging in a sexually explicit telephone conversation with a partner.

### RELATED APPLICATIONS

This application is the nonprovisional application of and claims priority to U.S. Patent Application Ser. No. 60/704,523, filed Aug. 1, 2005, and to U.S. Patent Application Ser. No. 60/703,983, filed Jul. 29, 2005, the disclosures of which are herein incorporated by reference.

### FIELD OF THE INVENTION

The present invention relates to sexual stimulation devices and toys that include features for playing audio and/or video received from an external source, such as a cellular phone or other remote device.

### BACKGROUND OF THE INVENTION

Dildos, vibrators, and other sex devices and toys are distributed throughout the world generally in sexual novelty shops and through Internet merchants. These devices can be used as adult sexual aids and/or given merely as gag gifts, for example, at an office party to embarrass the recipient. Usually, however, their primary purpose is that of a sexual stimulant.

Dildos and vibrators are usually phallic shaped so as to resemble the form (if not the size) of the penis or phallus. Typically, the term “dildo” generally refers to and includes phallic-shaped devices that are static or non-vibrating, while the term “vibrator” generally refers to and includes phallic-shaped devices that include a system for causing vibration of the device. But vibrators can also be shaped in other ways besides phallic shaped, and/or be provided such that they are not anatomically correct.

### SUMMARY OF THE INVENTION

According to various aspects of the present invention, sexual stimulation devices and sex toys, such as vibrators, include means (such as BLUETOOTH® technology, and the like) for communicating with (such as receiving and/or transmitting) and playing audio and/or video from a remote source, such as a cellular phone, a landline-based phone, the Internet, a personal computer, a cable network, a satellite network, a radio, a television, among other possible remote sources. In one particular aspect, a vibrator includes a BLUETOOTH® module configured to be able to link to a BLUETOOTH® enabled device, such as a cellular phone or a Voice Over IP (VOIP) computer. Aspects of the invention also include methods of using a device of the present invention. In one exemplary implementation, a device of the present invention is used for sexual stimulation while engaging in a sexually explicit telephone conversation with a partner.

Further aspects and features of the present invention will become apparent from the detailed description provided hereinafter. It should be understood that the detailed description and specific examples, while indicating exemplary aspects of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of a BLUETOOTH®-enabled vibrator including an end cap having an audio system and a BLUETOOTH® device for receiving audio and/or video from an external source according to one aspect of the present invention;

FIG. 2 is a perspective view of the BLUETOOTH®-enabled vibrator shown in FIG. 1 with a portion of the housing removed to illustrate the batteries, vibration-generating motor, and threaded engagement between the end cap and the housing;

FIG. 3 is a block diagram illustrating the audio system, the BLUETOOTH® device, and control circuitry for controlling one or more operations of a BLUETOOTH®-enabled vibrator according to one aspect of the invention; and

FIG. 4 is a block diagram of an exemplary system and method illustrating exemplary remote sources from which a BLUETOOTH®-enabled vibrator can receive audio and/or video according to one aspect of the invention.

### DETAILED DESCRIPTION

The following description of the various aspects of the invention is merely exemplary in nature and is in no way intended to limit the invention, its applications, or uses. In addition, the teachings of the present invention can be applied in a wide range of devices, including non-vibrating devices and vibrating devices outside of the sex industry. For example, aspects of the invention can be applied to vibrators, vibrating dildos, static or non-vibrating dildos, replica vaginas, replica butts/bottoms, inflatable or blow-up female dolls, inflatable or blow-up male dolls, sexual devices that are not anatomically correct (such as a pencil-type vibrator, bullet-type vibrator, rabbit-type vibrator, over-sized vibrators, double or triple headed vibrators, and the like) among other sexual stimulation devices, sex toys, and other devices. In addition, aspects of the invention can also be applied to devices and vibrating products that are not typically considered sex toys or sexual stimulation devices, but may nevertheless be used for sexual stimulation, such as back massagers and vibrating wands. Accordingly, the specific references herein to sexual devices, such as vibrators, should not be construed as limiting the scope of the present invention to any specific form/type of sexual toy or device.

According to various aspects of the present invention, sexual stimulation devices and sex toys, such as vibrators, include means (such as BLUETOOTH® technology) for communicating with (receiving and/or transmitting, and the like) and playing audio and/or video from a remote source, such as a cellular phone, a landline-based phone, the Internet, a personal computer, a cable network, a satellite network, a radio, a television, among other possible remote sources. In one particular aspect of the invention, a vibrator includes a BLUETOOTH® module configured to link to a BLUETOOTH® enabled device, such as a cellular phone or a Voice Over IP (VOIP) computer. Aspects of the invention also include methods of using a device of the present invention. In one exemplary implementation, a device of the present invention can be for sexual stimulation while also being used to carry on a sexually explicit telephone conversation with another.

According to various aspects of the present invention, sexual stimulation devices and toys (of which a vibrator is just one example) include means (such as BLUETOOTH® technology) for communicating with (such as, receiving and/or transmitting) a remote or external source (such as BLUETOOTH® enabled communication device, such as a cellular phone or Voice Over IP (VOIP) computer). The device also includes means for playing audio and/or video received from that remote source. Exemplary remote sources include cellular phones, land-based phones, the Internet, a personal computer (such as, VOIP enabled computer), a cable network, a satellite network, radio communications, television broadcasts, among other possible remote sources.

According to various aspects of the invention, the present invention includes vibrators having a mechanism capable of playing prerecorded audio and/or custom audio (such as, personalized voice messages).

Another aspect of the invention relates to methods of sexual stimulation that generally include sexually explicit telephone calls (such as telephone sex) with another, such as a lover or partner who is in a remote location. Various aspects described herein combine a vibrator and a method of having telephone sex into one device/application capable of both. A vibrator can include BLUETOOTH® technology or other suitable technology for linking to a cellular phone, in which case, the user can concurrently use the vibrator as a sexual stimulant while simultaneously having a sexually explicit telephone conversation (such as telephone sex).

A vibrator typically includes a BLUETOOTH® module configured to link to a BLUETOOTH® enabled device, such as a cellular phone or a Voice Over IP (VOIP) enabled computer. Additionally, or alternatively, other aspects include a vibrator having BLUETOOTH® or other suitable technology such that the vibrator is capable of both receiving and initiating telephone calls. A vibrator can further include one or more speakers for listening to audio messages and/or one or more microphones for transmitting audio messages.

In other aspects of the invention, vibrators can be linked via BLUETOOTH® technology (or other suitable technology) with a medium such as a radio, television and/or personal computer so that audio and/or video can be transmitted to and then played by the vibrator as an additional stimulant. A vibrator according to these aspects may be linked with background music, other audio, and/or visual messages as these messages could be played through the vibrator in order to generally enhance the sexual experience. By virtue of a BLUETOOTH® link or other communication link one could play the user's favorite music, sexually explicit transmissions, other audio/video programs, combinations thereof, and the like.

A BLUETOOTH® module may be incorporated into a traditional vibrator, such as that found in most adult novelty stores. This would result in a BLUETOOTH®-enabled vibrator. A BLUETOOTH®-enabled vibrator may also be produced by incorporating a BLUETOOTH® module into a more advanced audio enhanced vibrator version that is capable of playing prerecorded audio and/or custom audio (for example, personalized voice messages, and the like), such as one or more of the devices described in pending U.S. application Ser. No. 11/081,050. In one aspect of the invention, the BLUETOOTH® module is of the type capable of being integrated with a cellular phone. According to other aspects, any one or more of the devices described in pending U.S. application Ser. No. 11/081,050 can be provided with a BLUETOOTH® module, or other suitable communication module. The contents of U.S. application Ser. No. 11/081,050 are herein incorporated by reference.

An exemplary use of one of the devices of the present invention will now be described for purposes of illustration only. While generally in a secure place, a user may use one of the devices of the present invention to carry on a hands free sexually explicit telephone conversation (for example, telephone sex) with a partner (typically, a partner in a remote location.) while also simultaneously using that device (for example, a vibrator) to achieve sexual gratification. The user can carry on the telephone conversation by virtue of the BLUETOOTH® feature of the vibrator (or other sexual device) being linked to a BLUETOOTH® enabled communication device (for example, cellular phone, VOIP enabled computer, and the like), which, in turn, is linked to a partner's communication device (for example, the partner's cellular phone, VOIP enabled computer, landline telephone, and the like). By using a vibrator or other sexual device, a user can thus communicate with and carry on a more technologically advanced form of hands free telephone sex with the partner even when the partner is in a remote location, such as a soldier serving overseas. In one particular aspect, a BLUETOOTH® module is incorporated into or disposed within the body of the vibrator and/or the vibrator's end cap, such that the combination of the vibrator and BLUETOOTH® module can function as one unit wherein the BLUETOOTH® module allows the user to link to a cellular phone, which in turn, is linked to a lover's cellular phone. The BLUETOOTH® module may also allow the user to wirelessly use and administer one or more of the existing features and functions of the cellular phone, such as caller identification, auto dialing, voice-activated dialing, and the like.

In other aspects of the present invention, a vibrator (or other type of adult sexual aid.) can include a BLUETOOTH® module or other communication device that is capable of administering most of a phone's normal functions, such as both receiving and dialing out remotely. Further, the vibrator and/or an audio enhanced vibrator in various aspects can be further capable of all of the functions typically associated with a cellular phone. For example, the vibrator's user can dial out or receive a telephone call via a BLUETOOTH® device. The BLUETOOTH® function can further be expanded to such items as identifying which party to call via a voice command. Accordingly, these aspects can allow a user to carry on hands free telephone sex by using a vibrator (or other sexual device) as both a sexual stimulant and as a hands free cellular telephone. For example, a user could use a vibrator having a BLUETOOTH® module (or other suitable communication module) capable of initiating and/or receiving telephone calls in the context of having telephone sex with a partner, who may be in another location. In one particular aspect, a BLUETOOTH® module is incorporated into or disposed within the body of the vibrator and/or the vibrator's end cap, such that the combination of the vibrator and BLUETOOTH® module can function as one unit, wherein the BLUETOOTH® module allows the user to have hands free telephone conversations while the vibrator concurrently serves as a sexual stimulant.

A vibrator may also include a BLUETOOTH® module (or other suitable communication module) that can be integrated and/or communicate with a radio, CD player, DVD player, television, personal computer, combinations thereof, and/or like medium. A vibrator may thus serve as both a sexual stimulant and an entertainment center, thereby further enhancing the sexual experience through some additional form of audio and/or video stimulus.

Any of the aspects of the present invention may be used individually or in combination with any one or more of the other aspects of the invention. In addition, further aspects and features of the present invention will become apparent from the description herein. It should be understood that the description and specific examples herein, while indicating exemplary aspect of the invention, are intended for purposes of illustration only and are not intended to limit the scope of the invention.

Referring now to FIGS. 1 through 3, there is shown a vibrator, generally indicated by reference number 100 according to one aspect of the present invention. As shown, the vibrator 100 includes a housing 102 and an end cap 104. The vibrator 100 also includes a BLUETOOTH® module or device 122. Alternatively, the vibrator 100 can include another suitable communication device or technology besides BLUETOOTH® that is capable of transmitting and/or receiving signals from an external source, via cellular phone transmissions, wireless signals or transmissions communicated between antennae, electromagnetic wave energy, RF energy, radio transmissions or communications (for example, citizens band (CB), and the like), satellite transmissions, television transmissions, walkie-talkie transmissions, CD player, DVD player, television, personal computer, and combinations thereof.

In the particular illustrated aspect, the outside of the housing 102 is generally phallic-shaped so as to resemble the form (if not the size) of a human penis. The housing 102 can be provided in various sizes and colors, some which are anatomically correct and others that are not anatomically correct. In addition, a wide range of materials may be used for the housing 102, including rubber, gel foams, plastics, silicone, and other suitable materials. The housing 102 may be configured (for example, sized, shaped, colored, formed of a material, and the like) in a manner similar to existing commercially available dildos and vibrators. As described herein, various aspects include an end cap that is retrofit onto an existing vibrator. Again, however, aspects and teachings of the present invention are not limited to phallic-shaped vibrators.

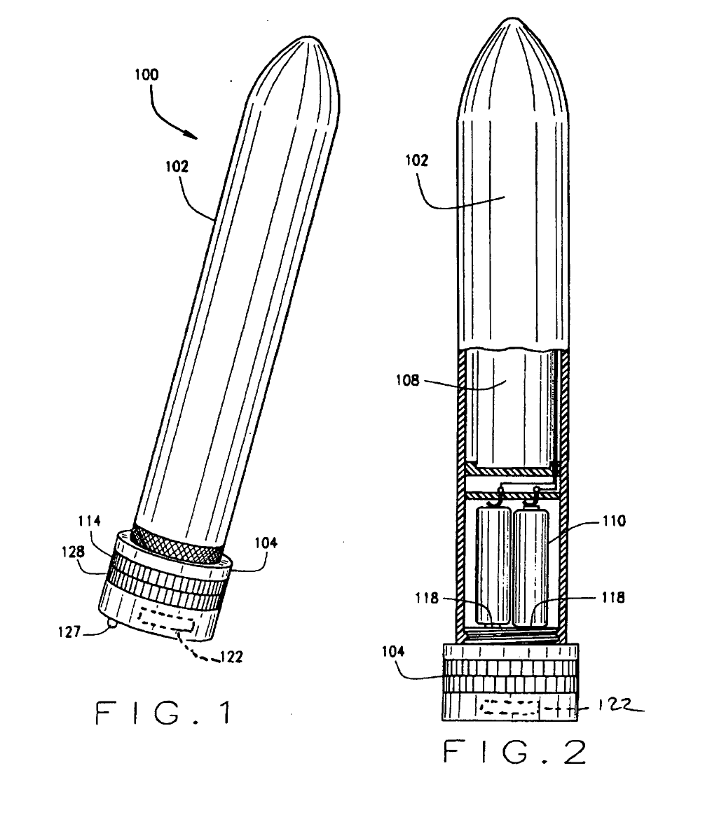

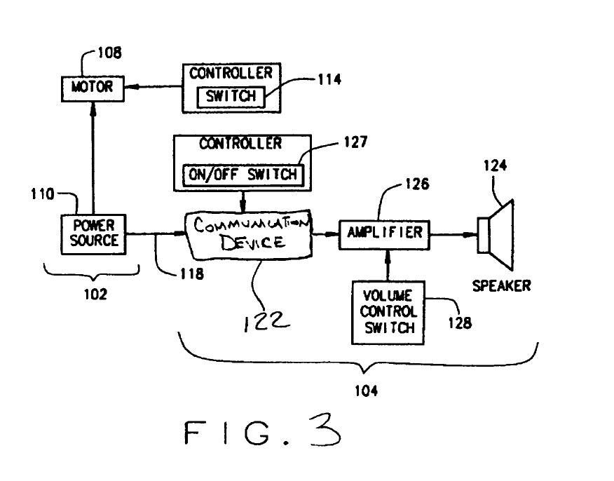

As shown in FIG. 2, the housing 102 defines a generally hollow cavity in which is positioned a motor 108 and batteries 110 for providing electrical power for operating the motor 108. When electrically connected to the batteries 110, the motor 108 causes the housing 102 to vibrate. Accordingly, the vibrator 100 of this aspect is a portable handheld “vibrator”. Alternatively, other aspects do not include a vibration-causing motor, such as a static or non-vibrating vibrator or dildo.

FIG. 2 illustrates two AA batteries 110 positioned with the housing 102. Alternatively, other suitable power sources can be used for providing the electrical power to the motor 108, including other battery types, battery sizes, renewable batteries, rechargeable batteries, disposable batteries, and the like. In some aspects, the motor 108 can receive electrical power from a power source external to the housing 102. For example, the housing 102 may include an AC adapter port into which an electrical cord is plugged such that the motor 108 receives electrical power through the electrical cord from a standard wall outlet. Or, for example, the motor within the housing can receive electrical power via connectors from one or more batteries within the end cap.

With further reference to FIGS. 1 through 3, the vibrator 100 includes at least one switch for controlling operation of the motor 108. As shown in FIG. 3, the vibrator 100 includes switch 114 for switchably connecting the motor 108 to the batteries 110. The switch 114 may also allow the user to adjust the rate of speed and/or manner of vibration. Alternatively, the vibrator may include a first “ON/OFF” switch for activating and deactivating the vibration-generating motor, and a second “variable speed switch” switch for controlling the rate and/or type of vibration. In other aspects, the vibration may be constant such that the switch 114 is only used to turn on or turn off the vibration features.

In addition, the invention may include the switch 114 being used to control the operation of the audio features as well. That is, the manner in which the operation of the vibration feature is controlled may also be used for controlling operation of the audio system. Alternatively, and as shown in the figures and described herein, there may be separate (or at least partially separate) systems for controlling the operation of the audio features and the vibration feature. Various aspects of the invention include a means (for example, switch 114) for controlling operation of the vibration feature that is entirely independent and distinct from the means (for example, on/off push-button switch 127, volume control switch 128) for controlling operation of the audio features and the communication device 122. As shown in FIG. 3, the switch 114 can be used to control the operation of the motor 108, and the push-button switch 127 can be used to control the communication device 122 (for example, turn it off/on) and audio features.

By allowing a user to control the operation of the vibration independent of the audio, various aspects advantageously allow the user to independently adjust the audio and the vibration (and noise created thereby) so that the user can still hear the audio while using the vibrating device. For example, the user can increase the volume of the audio and/or decrease the intensity of the vibration so that the noise created by the vibration does not drown out the audio.

A “panic” switch may be provided for pausing and/or stopping the audio and/or video play. This stop switch may be activated, for example, when an unexpected interruption occurs (for example, an unexpected visitor knocking on the front door). Activating the panic switch may pause or stop the play in a manner such that the user can return to the same place in the audio and/or video without having to replay the entire audio and/or video and without having to start all over from the beginning. Activating the panic switch may also pause the audio and/or video play and also deactivate and shut off the vibration.

As shown in FIGS. 1 through 3, the end cap 102 includes the switch 114 for controlling operation of the motor 108. In the illustrated aspect, the switch 114 is a rotationally adjustable annular ring that is provided on the end cap 104. By rotating the ring relative to a longitudinal centerline axis of the device 100, the user can control the operation of the motor 108. For example, the user can rotate the ring to turn on/off the motor 108, and/or adjust the vibration speed (increase or decrease) depending on the direction and amount of rotation of the ring 114. Alternatively, the rotationally adjustable ring 114 can be disposed around a portion of the housing 102. In addition, other suitable switches can be implemented for controlling operation of the motor 108, including push-button switches, electrical terminals (such as externally disposed on the end cap 104 or housing 102) for switchably connecting the motor 108 to the batteries 110 when the electrical terminals are placed in contact with the user's body, motion-responsive switches, light-sensitive switches, temperature-sensitive switches, compression switches, voice activated switches, and the like. For example, a push-button switch may be provided on the end cap and/or the housing that allow the user to turn on/off the vibration feature and/or cycle through various vibration modes by successively pressing the push-button switch.

The end cap 104 and its audio system will now be described. As shown, the end cap 104 is threadably engaged to the housing 102. Accordingly, the end cap 104 can be readily removed from the housing 102 if the user wants to access the cavity 106 within the housing 102, for example, to replace the batteries 110. Alternatively, other suitable fastening methods can be employed for removably engaging the end cap 104 to the housing 102. The end cap 104 may be fixedly attached to or integral with the housing 102. A waterproof seal is typically formed between the end cap 104 and housing 102 when the end cap 104 is engaged to the housing 102.

The end cap 104 includes an audio system and the communication module 122. The end cap 104 also includes connectors 118 for switchably connecting the audio system and communication device 122 to the batteries 110 when the end cap 104 is threadably engaged to the housing 102. As shown in FIG. 2, two AA batteries 110 are positioned with the housing 102. Alternatively, other suitable power sources may be used for providing the electrical power to the audio system and communication device 122, including other battery types, battery sizes (for example., watch batteries, and the like), renewable batteries, rechargeable batteries, and disposable batteries. The motor 108, audio system, and communication device may also receive electrical power from different power sources.

In another aspect, an end cap includes batteries for providing electrical power for operating the audio system, the vibration-generating motor, and the BLUETOOTH® module. Yet another aspect includes an end cap having batteries for providing electrical power for operating the audio system, but the vibration-generating motor and/or BLUETOOTH® module receives electrical power from a separate set of batteries within the housing. The audio system, vibration-generating motor and/or BLUETOOTH® module may receive electrical power from a power source external to the vibrator. As yet another example, the end cap may include an AC adapter port into which an electrical cord is plugged such that the audio system, vibration-generating motor and/or BLUETOOTH® module receives electrical power through the electrical cord from a standard wall outlet.

The audio system includes means for emitting sound (for example, voice messages, music, instructions, or other sounds) corresponding to audio data received by the BLUETOOTH® module 122. As shown in FIG. 3, the means for emitting sound comprises amplifier 126 and speaker 124.

As shown in FIGS. 1 through 3, the vibrator 100 also includes a BLUETOOTH® device 122. The vibrator 100 also includes a controller for controlling the operation of the BLUETOOTH® device 122, such as a controller for turning it off and on, among other possible control features.

As shown, the BLUETOOTH® device 122 is generally housed in the end cap 104 of the vibrator 100. Alternatively, the BLUETOOTH® device can be located at other suitable vibrator locations, such as with the housing 102, and the like.

In one particular aspect, the vibrator 100 via the BLUETOOTH® module 122 can be linked to a cellular phone. The BLUETOOTH® module 122 may allow the user to wirelessly use and administer one or more of the existing features and functions of the cellular phone, such as caller identification, auto dialing, voice-activated dialing, and the like.

Advantageously, the user can use the vibrator 100 to maintain a telephone conversation while simultaneously using the vibration feature for sexual gratification. In effect, the user now has one device that is capable of all of the typical vibrator functions as well as remotely maintaining a telephone conversation by virtue of the BLUETOOTH® feature linked to a BLUETOOTH® enabled communication device (for example, cellular phone, Voice Over IP (VOIP) computer, and the like).

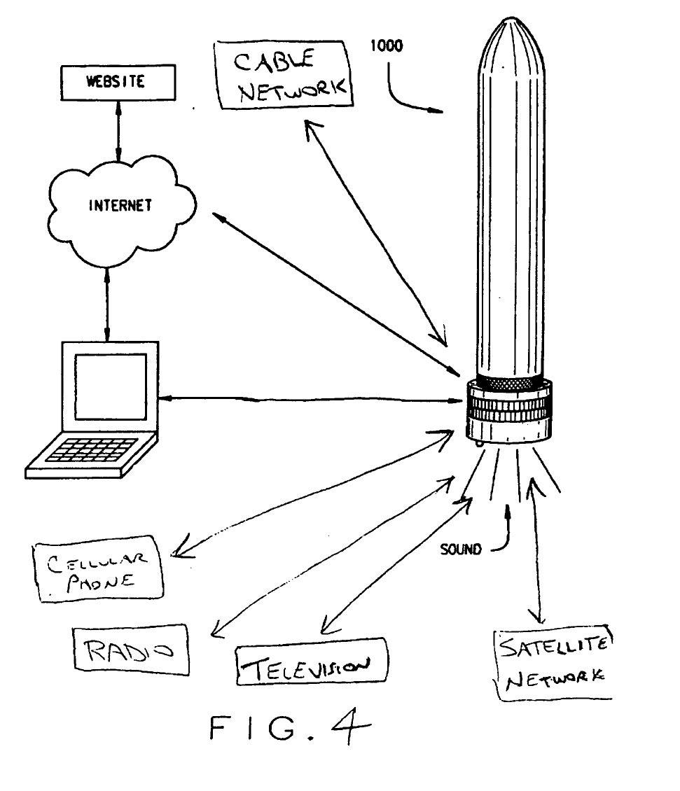

FIG. 4 illustrates an exemplary vibrator 1000 having a BLUETOOTH® or other communication module. As shown, the vibrator 1000 is linked to or communicating with (for example, receiving and/or transmitting data) one or more remote devices. By way of example only, such remote devices (as shown in FIG. 15) may include the Internet, a personal computer, a cellular phone, a radio, a television, a satellite network, a cable network, combinations thereof, and the like. The vibrator 1000 may be configured to communicate with only one of such remote devices, particular combinations of such remote devices, and the like.

Another aspect of the invention relates to the BLUETOOTH® technology and its use for audio enhancement and/or video enhancement by linking via BLUETOOTH® or like technology to a medium that is capable of transmitting audio, video, or some combination of audio and video like a television set. The user may remotely play, for example, an adult-themed movie and hear the movie's dialogue and other sounds through a link established between the vibrator and television set (or other medium). The BLUETOOTH® or like technology in this example adds further stimulation to the experience by virtue of the sound emitted through the vibrator's link to a medium, such as a television set.

It is anticipated that products will be sold in distribution systems related to sexual novelty devices, such as adult novelty stores or by way of the Internet. The product may be targeted at those customers in the market for sexual devices, such as vibrators, as aspects of the invention can be a substitute or an enhancement to a sexual vibrator in the context of engaging in telephone sex, among other uses. Additionally, or alternatively, aspects of the invention may further be construed as an unusual gag gift, where features and aspects of the present invention can increase the commercial appeal thereof.

The vibration is typically responsive to the pitch, volume, or other attribute of the audio that is received and/or played by the vibrator (or other sexual device or toy). At least one attribute of the vibration (for example, intensity, modality, and combinations thereof) may be controlled as a function of at least one attribute of the audio (for example, pitch, intensity, rhythm, bass beats, decibels, volume level, wavelength, frequency, and combinations thereof). One exemplary aspect includes processing (such as translating) audio received and/or played by a vibrator such that the vibration intensity and/or modality changes (increases or decreases) as the intensity, pitch, and/or volume of the audio changes. In one particular aspect, the intensity and modality of the vibrations of a vibrator (such as a BLUETOOTH®-enabled vibrator, one or more of the devices described in pending U.S. application Ser. No. 11/081,050 and the like) are automatically controlled to translate to the intensity, pitch, and/or volume of the audio (such as, audio received via the Bluetooth module, and/or audio played by the vibrator) such that the vibration pulses and intensifies along with increases in the audio's intensity, pitch, and/or volume, and vice versa.

The teachings of the present invention can be applied in a wide range of devices, including non-vibrating devices and vibrating devices outside of the sex industry. For example, aspects of the invention can be applied to vibrators, vibrating dildos, static or non-vibrating dildos, replica vaginas, replica butts/bottoms, inflatable or blow-up female dolls, inflatable or blow-up male dolls, sexual devices that are not anatomically correct (such as pencil-type vibrator, bullet-type vibrator, rabbit-type vibrator, over-sized vibrators, double or triple headed vibrators, and the like) among other sexual stimulation devices and sex toys. In addition, aspects of the invention can also be applied to devices and vibrating products that are not typically considered sex toys or sexual stimulation devices, but may nevertheless be used for sexual stimulation, such as back massagers, vibrating wands, and the like. Accordingly, the specific references herein to sexual devices, such as vibrators, should not be construed as limiting the scope of the present invention to any specific form/type of sexual toy or device.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present invention, the articles “a”, “an”, “the” and “said” are intended to mean that there are one or more of such elements or features. The terms “comprising”, “including” and “having” are intended to be inclusive and mean that there may be additional elements or features other than those specifically noted.

Although illustrated aspects of the present invention are described herein with reference to the accompanying drawings, it is understood that the invention is not limited to those precise aspects that various other changes and modifications may be effected therein by one skilled in the art without departing from the scope or spirit of the invention. The scope of the invention is defined solely by the claims, and their equivalents, appended hereto.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A vibrator comprising a BLUETOOTH® module.

2. The vibrator of claim 1 wherein said BLUETOOTH® module is linked to a BLUETOOTH® enabled device.

3. The vibrator of claim 2, wherein the BLUETOOTH® module of the vibrator is configured to link to a BLUETOOTH® enabled device selected from the group consisting of a cellular phone, a Voice Over IP (VOIP) computer, and combinations thereof.

4. The vibrator of claim 1 wherein said BLUETOOTH® module is linked to a cellular telephone.

5. The vibrator of claim 1 wherein said vibrator is capable of initiating telephone conversations remotely.

6. The vibrator of claim 5 wherein said vibrator is linked to a cellular phone.

7. The vibrator of claim 1 wherein said vibrator is capable of receiving telephone conversations remotely.

8. The vibrator of claim 8 wherein said vibrator is linked to a cellular phone.

9. The vibrator of claim 1 wherein said vibrator is capable of initiating and receiving telephone calls.

10. The vibrator of claim 9 wherein said vibrator is linked to a cellular phone.

11. The vibrator of claim 9 wherein said vibrator comprises at least one speakers for listening to a received telephone call.

12. The vibrator of claim 5 wherein said vibrator comprises at least one microphone for transmitting live audio messages to a third party.

13. The vibrator of claim 5 wherein said vibrator comprises at least one speaker for listening to live audio messages from a third party and at least one microphone for transmitting live audio messages to a third party.

14. The vibrator of claim 7 wherein said vibrator comprises at least one speaker for listening to live audio messages from a third party and at least one microphone for transmitting live audio messages to a third party.

15. The vibrator of claim 9 wherein said vibrator is capable of initiating telephone calls from a remote location, and wherein said vibrator comprises one or more microphones for transmitting live audio messages to a third party in another remote location.

16. The vibrator of claim 9 wherein said vibrator is capable of receiving telephone calls from a remote location, and wherein said vibrator comprises one or more speakers for listening to telephone messages from a third party at another remote location.

17. The vibrator of claim 9 wherein said vibrator is capable of receiving and initiating telephone calls from a remote location, and wherein said vibrator comprises one or more speakers for listening to live audio messages from a third party in another remote location and one or more microphones for transmitting live audio messages to a third party in another remote location.

18. The vibrator of claim 9 wherein said vibrator is capable of initiating telephone calls from a remote location, and wherein said vibrator is capable of identifying the recipient of the telephone call via audio instructions.

19. The vibrator of claim 9 wherein said vibrator is capable of causing a BLUETOOTH® enabled cellular phone to initiate telephone calls from a remote location, and wherein said vibrator is capable of identifying the recipient of the telephone call via audio instructions.

20. The vibrator of claim 18, wherein said audio instructions are selected from the group consisting of identifying a telephone call by a specific person's name, and identifying a telephone call by the last number dialed.

21. The vibrator of claim 18 wherein said vibrator is capable of identifying to whom the call will be placed via audio instructions that will initiate a numerical sequence in the context of placing the telephone call.

22. The vibrator of claim 19 wherein said vibrator is capable of causing a BLUETOOTH® enabled cellular phone to receive telephone calls from a remote location and to identify the person who has initiated the call via an audio message.

23. The vibrator of claim 21, wherein the audio message includes the person's name.

24. The vibrator of claim 9 wherein said vibrator is capable of identifying the person who has initiated the call via the initiating party's telephone number.

25. The vibrator of claim 9 which further comprises a video monitor integral to the vibrator which is capable of receiving video transmissions.

26. The vibrator of claim 9 which further comprises a video monitor not integral to the vibrator which is capable of receiving video transmission.

27. A vibrator capable of transmitting video via a BLUETOOTH® link with a camera or video recorder.

28. A vibrator capable of filming video via a BLUETOOTH® link with a camera or video recorder.

29. The vibrator of claim 27 which further comprises a mechanism capable of transmitting a video integral to the vibrator via a BLUETOOTH® link with a camera or video recorder.

30. The vibrator of claim 28 which further comprises a mechanism capable of filming video integral to the vibrator via a BLUETOOTH® link with a camera or video recorder.

31. The vibrator of claim 9 wherein said vibrator is capable of receiving remote audio via a BLUETOOTH® link to a radio.

32. The vibrator of claim 9 wherein said vibrator is capable of receiving remote audio via a BLUETOOTH® link to a CD player.

33. The vibrator of claim 9 wherein said vibrator is capable of receiving remote audio via a BLUETOOTH® link to a DVD player.

34. The vibrator of claim 9 wherein said vibrator is capable of receiving remote audio via a BLUETOOTH® link to a television.

35. The vibrator of claim 9 wherein said vibrator is capable of receiving remote audio via a BLUETOOTH® link to a personal computer.

36. The vibrator of claim 9 wherein said vibrator is capable of receiving remote audio via a BLUETOOTH® link to a cable network.

37. The vibrator of claim 9 wherein said vibrator is capable of receiving remote audio via a BLUETOOTH® link to a satellite network.

38. The vibrator of claim 9 wherein said vibrator is capable of receiving remote video via a BLUETOOTH® link to a television.

39. The vibrator of claim 9 wherein said vibrator is capable of receiving remote video via a BLUETOOTH® link to a personal computer.

40. The vibrator of claim 9 wherein said vibrator is capable of receiving remote video via a BLUETOOTH® link to a cable network.

41. The vibrator of claim 9 wherein said vibrator is capable of receiving remote video via a BLUETOOTH® link to a satellite network.

42. The vibrator of claim 9 wherein said vibrator is capable of receiving remote video via a BLUETOOTH® link to a DVD player.

43. The vibrator of claim 9 wherein said vibrator is capable of receiving remote video via a BLUETOOTH® link with a monitor integral to the vibrator for viewing the video transmission.

44. The vibrator of claim 9 wherein said vibrator is capable of receiving remote video via a BLUETOOTH® link with a monitor not integral to the vibrator for viewing the video transmission.

45. The vibrator of claim 9 wherein said vibrator is capable of receiving remote audio and remote video via a BLUETOOTH® link with a personal computer.

46. The vibrator of claim 9 wherein said vibrator is capable of receiving remote audio and remote video via a BLUETOOTH® link with. DVD player.

47. The vibrator of claim 9 wherein said vibrator is capable of receiving remote audio and remote video via a BLUETOOTH® link with a cable network.

48. The vibrator of claim 9 wherein said vibrator is capable of receiving remote audio and remote video via a BLUETOOTH® link with a satellite network.

49. A sexual device comprising a BLUETOOTH® module.

50. A sexual stimulation device comprising a BLUETOOTH® module.

51. The device of claim 50 wherein said BLUETOOTH® module is operably linked to a BLUETOOTH® enabled device.
